(19) United States
(12) Patent Application Publication
(10) Pub. No.: US 2010/0077493 A1
(43) Pub. Date: Mar. 25, 2010

(54) GENES AND PATHWAYS INVOLVED IN BIPOLAR DISORDER

(76) Inventor: Guoping Feng, Chapel Hill, NC (US)

Correspondence Address: MICHAEL BEST & FRIEDRICH LLP, 100 E WISCONSIN AVENUE, Suite 3300, MILWAUKEE, WI, 53202, US

(21) Appl. No.: 12/563,567

(22) Filed: Sep. 21, 2009

Related U.S. Application Data
(60) Provisional application No. 61/098,503, filed on Sep. 19, 2008.

(51) Int. Cl. G01N 33/50; A01K 67/027; C12Q 1/02; C12Q 1/48; C12Q 1/68; A61K 31/7088

(52) U.S. Cl. 800/3; 800/14; 800/18; 435/325; 435/29; 435/15; 435/6; 514/44 R

(57) ABSTRACT

Transgenic non-human mammals and cells having a disruption in at least one allele of nArgBP2 are provided. Methods of identifying therapeutic agents for the treatment of a disorder associated with altered expression of nArgBP2 are also provided. Methods of assessing the risk of an individual developing a disorder associated with disruption of nArgBP2 and methods of treating individuals with such a disorder are provided.

### CROSS-REFERENCE TO RELATED APPLICATIONS

This application claims the benefit of U.S. Provisional Patent Application No. 61/098,503 filed Sep. 19, 2008, which is incorporated by reference herein.

### STATEMENT REGARDING FEDERALLY SPONSORED RESEARCH

This invention was made with government support under grant number, R01 NS42609, awarded by the National Institutes of Health. The government has certain rights in the invention.

### BACKGROUND

Bipolar disorder is one of the most common, severe and devastating neuropsychiatric disorders, affecting 3-5% of the population worldwide (Shastry, 2005, Neurochem Int. 46:273-9). It is the third leading cause of death among people aged 15-24. Despite the high prevalence and severity of bipolar disorder, remarkably little is known about its neurobiological basis. Neuroanatomical and functional imaging studies have implicated prefrontal cortex, striatum and amygdala in the pathogenesis of bipolar disorder (Hajek et al., 2005, Bipolar Disord. 7:393-403; Strakowski et al., 2005, Mol. Psychiatry. 10:105-16).

Many lines of evidence strongly suggest that bipolar disorder has a genetic basis. Concordance for bipolar disorder is greater among pairs of monozygotic twins (79%) than among pairs of dizygotic twins (19%). Family studies of bipolar disorder also indicate that the risk to first degree relatives is 10 times greater than that for the general population.

### SUMMARY OF THE INVENTION

In one aspect, transgenic non-human mammals including a disruption in at least one allele of nArgBP2 (also known as SORBS2) are provided. The transgenic non-human mammal has a phenotype distinct from that of a non-human mammal of the same species lacking a disruption in an allele of nArgBP2. Neuronal cells including a disruption in at least one allele of nArgBP2 are also provided. Neuronal cells from the transgenic non-human mammal are also provided.

In another aspect, methods of identifying a therapeutic agent for the treatment of a disorder are provided. In the methods, the level of a nArgBP2 activity or nArgBP2 expression in a cell is evaluated after contacting the cell with a test agent. The level of a nArgBP2 activity or nArgBP2 expression in the cell is then compared to that of a control. A change in the nArgBP2 activity or nArgBP2 expression in the cell as compared to the control is indicative of effectiveness of the test agent to treat the disorder.

In yet another aspect, methods of identifying a therapeutic agent for treatment of a condition associated with a disruption of nArgBP2 are disclosed. First, the test agent is administered to a subject with a disruption of at least one nArgBP2 allele. Then, a phenotype associated with disruption of the nArgBP2 allele in the subject after treatment with the test agent is compared to a control. A therapeutic agent for the treatment of the condition in a subject may be identified by the ability of the test agent to cause a change in the phenotype.

In still another aspect, methods of assessing the risk of an individual developing a clinical disorder associated with a disruption of nArgBP2 are provided. The method includes evaluating the nArgBP2 genotype or the expression of nArgBP2 in the individual, and then comparing the nArgBP2 genotype or the level of expression of nArgBP2 in the individual to that of a control. An altered nArgBP2 genotype or altered expression of nArgBP2 as compared to the control is indicative of the risk of developing a clinical disorder.

In a further aspect, methods of treating an individual with a disorder are disclosed. The methods include administering an effective amount of an enhancer of a nArgBP2 activity to the individual to ameliorate the disorder.

### BRIEF DESCRIPTION OF THE DRAWINGS

FIG. 1 is a set of photographs showing the results of an in situ hybridization for nArgBP2 mRNA. The photographs demonstrate expression of nArgBP2 in the cortex (CTX), amygdala (AMG) and the striatum (STR).

FIG. 2A is a cartoon depiction of the gene structure of nArgBP2 showing the 20 exons and the deletion of the brain specific exon to generate the nArgBP2 knock-out (KO) mouse.

FIG. 2B is a photograph showing the results of a PCR demonstrating the generation of heterozygous and homozygous nArgBP2 knockout mice.

FIG. 2C is a photograph of a Southern blot analysis of genomic DNA from wild-type

(WT) and gene targeted mouse embryonic stem cells showing disruption of nArgBP2 in one allele of the knockout mouse.

FIG. 2D is a photograph of a Western blot analysis showing the lack of nArgBP2 protein in brain lysate, synaptosomal membrane (SPM) and postsynaptic density (PSD) preparations from nArgBP2 mutant mice (KO). The same blot was reprobed with an anti-β-tubulin antibody as a loading control.

FIG. 3 is a set of graphs comparing the behavior of wild-type and nArgBP2 knockout mice. FIG. 3A-B demonstrates that nArgBP2 KO mice show increased activity in the open field test. FIG. 3C-G shows that nArgBP2 KO mice show risk-taking behaviors. FIG. 3H-I demonstrates that the risk-taking behaviors of nArgBP2 KO mice are not due to the altered level of anxiety since their behavior in the dark-light emergence test is similar to that of WT mice. FIG. 3J demonstrates that nArgBP2 KO mice show much less depression-like behavior as indicated by the dramatically reduced time of immobility in the tail suspension test. In FIG. 3, *p<0.05; **p<0.01.

FIG. 4 is a set of graphs showing the fearlessness of nArgBP2 knockout mice as compared to wild-type mice. FIG. 4A demonstrates that WT and nArgBP2 KO mice respond equivalently to foot shock. FIG. 4B-C demonstrates the reduced fear conditioning of nArgBP2 KO mice.

FIG. 5 is a set of graphs depicting the reduction of mania-like behaviors of nArgBP2 KO mice after treatment with lithium (LiCl) as compared to WT mice.

FIG. 6 is a set of graphs depicting the increase in weight and average food intake in the nArgBP2 KO mice.

FIG. 7 is a set of graphs showing the altered synaptic transmission at cortico-striatal synapsis in the nArgBP2 KO mice as compared to wild-type controls. FIG. 7A is a graph demonstrating that nArgBP2 KO mice exhibit normal cortico-striatal field potentials (total fEPSPs). FIG. 7B is a graph demonstrating the normal relationship between stimulation intensity and NP1 amplitude in nArgBP2 KO mice. FIG. 7C is a graph showing an increase in the NMDA receptor fEPSP peak in nArgBP2 KO mice. FIG. 7D is a graph showing an increase in the NMDA receptor fEPSP area in nArgBP2 KO mice.

FIG. 8 is a graph showing the altered synaptic NMDA and AMPA receptor compositions in the nArgBP2 KO mice as compared to wild-type mice. Data are shown as relative levels in nArgBP2 KO mice as compared to wild-type controls. β-actin and β-tubulin served as loading controls. The * indicates p<0.05 in a two-tailed t test.

FIG. 9A and FIG. 9B are graphs showing the circadian period length and the circadian period variation, respectively, of control WT and nArgBP2 KO mice when maintained in a dark environment after being raised under 12 hour light-12 hour dark cycles.

FIG. 9C and FIG. 9D are activity traces of individual WT and nArgBP2 KO mice when switched to a dark-dark cycle (arrows in C and D indicate the switch to dark-dark cycle).

### DETAILED DESCRIPTION

As described in the Examples, a genetic approach was used to study synaptic development and dysfunction in mice. The Examples demonstrate that nArgBP2, a postsynaptic protein at glutamatergic synapses in the brain, may play an important role in bipolar disorder. nArgBP2 was originally identified as a protein that directly interacts SAPAP3 in a yeast two-hybrid screen. SAPAP3 is a postsynaptic protein that interacts with PSD95 and Shank families of proteins to form the PSD95-SAPAP-Shank scaffolding complex, which is critical for the assembly and function of glutamatergic synapses. However, the function of nArgBP2 at the synapse is completely unknown and there are no previous in vivo studies of nArgBP2.

As shown in Example 1, nArgBP2 is highly expressed in brain regions that are strongly implicated in bipolar disorder, including cortex, striatum and amygdala. Transgenic mice with a deletion in the neuron specific exon of nArgBP2 were generated to analyze the function of nArgBP2 in vivo as described in Example 2. As demonstrated in Examples 3 and 4, genetic deletion of nArgBP2 in mice leads to mania/bipolar-like behavior including but not limited to increased activity, compulsive/repetitive behavior, risk-taking behavior, hedonistic behavior and anti-depressant-like behavior, resembling many aspects of symptoms in bipolar disorder patients. Remarkably, all these behavioral defects were corrected by treatment with lithium as shown in Example 5. In addition, as demonstrated in Example 6, nArgBP2 mutant mice are obese, a common problem with bipolar patients. Examples 7 and 8 demonstrate that nArgBP2 knock-out mice had altered synaptic transmission at the cortico-striatal synapses and had altered NMDA and AMPA receptor compositions at the synapses. Finally, Example 9 demonstrates that nArgBP2 KO mice have altered circadian patterns, another common problem in individuals with bipolar disorder.

The human nArgBP2 gene maps to a region on chromosome 4q35 which has a strong linkage to bipolar disorder. The mouse nArgBP2 nucleotide sequence is included as SEQ ID NO: 1. The nucleotide sequence contains several large introns and is alternatively spliced. The coding sequence is included as SEQ ID NO: 2. The mouse amino acid sequence is included as SEQ ID NO:3. The neuron specific exon is found at nucleotides 287,935-289,702 of SEQ ID NO: 1 and nucleotides 1781-2779 of SEQ ID NO:2. The particular deletion made to generate the knockout mouse in Example 2 was insertion of a STOP codon in the neuron specific exon of nArgBP2. Insertion of the STOP codon blocks production of the brain specific form of nArgBP2 in the mouse to produce a brain specific nArgBP2 KO mouse. The sequences are available in GenBank. The GeneID numbers for human and mouse nArgBP2 are 8470 and 234214, respectively. The human nucleotide sequence of nArgBP2 is provided as SEQ ID NO: 4. The human coding sequence is provided as SEQ ID NO: 5.

Transgenic non-human mammals with a disruption in nArgBP2 are described. Transgenic non-human mammals include all non-human mammals having a disruption in the nArgBP2 gene. Disruptions in nArgBP2 include any alteration to nArgBP2 which results in altered expression of nArgBP2 as compared to control healthy subjects. Altered expression includes both decreases and increases in expression. In particular, such altered expression may result in expression of a phenotype as described below. Suitably mice are the transgenic non-human mammal. The resulting transgenic animals may be called knockout mice. The production of nArgBP2 knockout mice or other knockout mammals can be carried out in view of the disclosure provided herein. Knockout mice may be generated using a variety of different techniques, such as those known to those skilled in the art. Mice having a mutation in the neuronal exon of nArgBP2 are disclosed in the examples below. The skilled artisan will also realize that a nArgBP2 deficiency can be produced in other animal species to generate other transgenic animals with altered nArgBP2 expression. Those skilled in the art will also appreciate that nArgBP2 protein levels may be affected using other techniques such as RNAi. Cells, tissues, organs, and progeny derived from the nArgBP2 deficient animal are also encompassed and described herein.

A knockout mouse is a mouse that contains within its genome a specific gene that has been inactivated by disruption, for example, by deletion, insertion or gene targeting. A knockout mouse includes both the heterozygote mouse (having one disrupted allele and one undisrupted allele) and the homozygous mutant having two defective alleles. Gene targeting is a type of gene recombination that occurs when a fragment of genomic DNA is introduced into a cell such that that fragment locates and recombines with endogenous homologous sequences. Such recombination can be as a replacement for deleted sequences but also can be an insertion of additional DNA. One important example of such insertion is the use of antibiotic resistance genes, such as the Neomycin/G418 resistance gene. Antibiotic resistance genes are well known and useful in helping select for modified cells with the appropriate insertion. A further example of deletion in the nArgBP2 gene is the deletion of one or more of the exons of the nArgBP2 gene. In the examples, the neuron specific exon of nArgBP2 is deleted. Disruption of nArgBP2 also includes, but is not limited to, nonsense mutations, single base substitutions, missence mutations or any other genetic alteration which results in aberrant expression of the nArgBP2 polypeptide as compared to the wild-type.

Gene disruption can result from a deletion of a portion of an endogenous gene or deletion of the complete gene. These substitutions and deletions can cause the protein encoded by the nArgBP2 gene to be expressed incorrectly (and thus not function properly) or to not be expressed at all. In another embodiment the disruption in nArgBP2 results in reduced expression of a functional nArgBP2 protein as compared to animals lacking the disruption. Levels of protein expression can be quantitatively assessed by a variety of methods known to those skilled in the art including, but not limited to, Western blots, protein assays and functional enzymatic assays.

The knockout mouse disclosed herein exhibits one or more phenotypes that distinguish the knockout mouse from a control mouse that does not have a deletion in nArgBP2. Control mice express functional nArgBP2 and exhibit the control phenotype. Comparison of the nArgBP2 deficient mouse to the control mouse was made under conditions wherein all mice were raised and maintained in the same environment under the same conditions. In the examples below, mice with a disruption of the neuron specific exon of nArgBP2, such that the mice have reduced nArgBP2 expression, exhibit any one, or any combination, of the following phenotypes: increased activity, compulsive behavior, risk-taking behavior, hedonistic behavior, obesity, fearless behavior, psychosis, repetitive behavior, irritable behavior, altered circadian patterns and anti-depressant-like behavior.

The phenotype of the knockout mouse of the presently disclosed subject matter indicates that the nArgBP2 protein plays a significant role in affecting the expression or pattern of phenotypes associated with bipolar disorder. Without being bound by theory, the nArgBP2 protein is thought to be involved in transducing/regulating signaling and structural changes at the post-synaptic site. In the Examples below the nArgBP2 knockout mice were shown to have altered synaptic NMDA and AMPA receptor compositions and increased synaptic transmission at cortico-striatal synapses.

Importantly, results presented in the Examples below indicate that the absence of functional nArgBP2 in an individual contributes to one or more disorders. A disorder that is associated with or directly caused by a reduction or absence of nArgBP2 protein in the diagnosed patient is referred to herein as a nArgBP2 disorder. A nArgBP2 disorder can result from aberrant expression of a nArgBP2 gene or aberrant function of an expressed nArgBP2 protein. One of skill in the art would predict from the phenotype of the nArgBP2 knockout mouse that an individual with reduced or absent functional nArgBP2 is at risk for development of a number of clinical disorders and complications known to arise from synaptic abnormalities, including psychiatric disorders that are thought to arise through defective synaptic signaling, such as bipolar disorder. Causes of a nArgBP2 disorder are not limited to loss or decrease in function of the nArgBP2 gene or protein. Indeed, overproduction or over activity of nArgBP2 in an individual is also expected to produce a disorder which manifests itself as distinct from controls.

The phenotype associated with a disruption in nArgBP2 may be affected by treatment of the transgenic animal with therapeutic agents known to treat bipolar disorder, such as lithium. In general, treatment of the transgenic animal with a therapeutic agent effective for treating bipolar disorder results in the transgenic animal behaving more like a wild-type control animal. In some cases, treatment results in the normalization of all of the phenotypes associated with bipolar disorder in the nArgBP2 knockout animal. Treatment includes, but is not limited to, an amelioration of at least one of the symptoms associated with the bipolar disorder affecting the host, where amelioration is used in a broad sense to refer to at least a reduction in the magnitude of a phenotype associated with the disorders, e.g. symptom. As such, treatment also includes situations where the pathological condition, or at least symptoms associated therewith, are completely inhibited, such that the host no longer suffers from the condition, or at least the symptoms that characterize the condition. Therapeutic agents that act as enhancers of nArgBP2 expression or function are expected to result in treatment of the disorder.

Also provided are neuronal cells having a disruption in at least one allele of nArgBP2 or having altered nArgBP2 expression or functionality. The cells may be derived from the transgenic animal with a disruption in at least one allele of the endogenous nArgBP2 described above. Cells derived from a knockout mouse refer to cells either in the mouse itself or separated from the mouse and expressing the disruption in nArgBP2 as described herein. Those skilled in the art will appreciate the cells may be derived from transgenic animals using a variety of techniques. Such cells may be derived by harvesting embryonic neuronal cells from transgenic animals. These nArgBP2 knockout neuronal cells may be used for a variety of purposes apparent to those of skill in the art including but not limited to, cell based assays, molecular assays or intracellular assays, such as cell signaling assays. For example, the cells may be used to study the cellular basis for the phenotypic behaviors associated with bipolar disorder and the role of nArgBP2 in normal neuronal cells.

Methods of identifying a therapeutic agent for the treatment of a disorder are also provided herein. Briefly, the level of a nArgBP2 activity or nArgBP2 expression in a cell after contact with a test agent may be evaluated and the levels detected to assess any change caused by treatment with the agent. For example, the levels of nArgBP2 activity or expression may be compared to those in a control or to those in the cell prior to contact with the test agent. The cells may be contacted by any means known to those skilled in the art and include in vivo, in vitro and ex vivo methods. Cells within or derived from a nArgBP2 knockout mouse may be used. The cells may be neuronal cells. Control cells may be cells from a subject that does not have the disorder, such as cells from a control mouse lacking a disruption in nArgBP2. Control cells may also be cells not contacted with the test agent or prior to contact with the test agent. A change in the level of a nArgBP2 activity or nArgBP2 expression which causes the levels in the cell to become closer to those observed in a cell lacking an nArgBP2 disruption is indicative of the test agent being a therapeutic agent for the treatment of the disorder. The disorder may be any disorder associated with aberrant nArgBP2 activity or aberrant nArgBP2 expression, and includes but is not limited to, bipolar disorder, schizophrenia and autism.

A nArgBP2 activity includes, but is not limited to, any naturally occurring biological activity that a nArgBP2 gene product has in vivo. Exemplary nArgBP2 activities include, but are not limited to, interacting with members of the MAGUK (membrane associated guanylate kinase) family (e.g., PSD-95), SAPAP3, the SHANK family, kinases, kinase binding proteins, and others. Additional activities of nArgBP2 include those that nArgBP2 has in modulating the activity of cortico-striatal synapses. For example, an nArgBP2 activity includes, but is not limited to, a change in expression of SAPAP3, a change in SAPAP3 activity, a change in expression of NMDA receptors, a change in activity of NMDA receptors, a change in expression of AMPA receptors, a change in activity of AMPA receptors, a change in p21-activated kinase (PAK) activity, or a change in PAK activity.

A nArgBP2 disorder can arise from a number of potential genetic defects. Aberrant (reduced or increased) expression of wild type nArgBP2, possibly resulting from a mutation in nArgBP2 gene regulatory sequences, or a mutation in a regulatory protein of nArgBP2 gene expression or protein function is expected to lead to a nArgBP2 disorder. Alternatively, expression of a mutant (i.e. having at least one amino acid change as compared to the control nArgBP2 protein) nArgBP2 protein having impaired function would lead to a nArgBP2 disorder. All animals known to express nArgBP2 (e.g., rodents, canines, felines, equines, and primates, especially humans) can potentially have a nArgBP2 disorder, and can be diagnosed as such by the methods described below.

A therapeutic agent includes agents which have been shown to ameliorate the symptoms of bipolar disorder in a test subject. A test agent can be tested on a subject with bipolar disorder and then compared to another subject without bipolar disorder or compared to the subject before treatment. Where the test agent ameliorates the anxiety symptoms in the test subject the test agent can be considered a therapeutic agent. A test agent can also be considered a therapeutic agent where increases in the amount of nArgBP2 protein are produced in an individual. These agents may also be enhancers of nArgBP2.

Test agents or libraries of test agents include natural or synthetic organic compounds, including but not limited to oligomers, non-oligomers, or combinations thereof. Non-oligomers include a wide variety of organic molecules, such as heterocyclics, aromatics, alicyclics, aliphatics, and combinations thereof, comprising steroids, antibiotics, enzyme inhibitors, ligands, hormones, drugs, alkaloids, opioids, benzodiazepenes, terpenes, porphyrins, toxins, catalysts, as well as combinations thereof. Oligomers include peptides (that is, oligopeptides) and proteins, oligonucleotides (the term oligonucleotide is also referred to simply as “nucleotide”, herein) such as DNA and RNA, oligosaccharides, polylipids, polyesters, polyamides, polyurethanes, polyureas, polyethers, poly (phosphorus derivatives) such as phosphates, phosphonates, phosphoramides, phosphonamides, phosphites, phosphinamides, etc., poly (sulfur derivatives) such as sulfones, sulfonates, sulfites, sulfonamides, sulfenamides, etc., where for the phosphorous and sulfur derivatives the indicated heteroatom for the most part will be bonded to C, H, N, O or S, and combinations thereof.

Methods of identifying therapeutic agents for the treatment of subjects expressing a phenotype associated with a condition such as bipolar disorder, schizophrenia or autism are also provided. Briefly, test agents may be administered to subjects with the condition in which the subject has a disruption in at least one nArgBP2 allele or altered expression or activity of a nArgBP2 polypeptide. The phenotype of the subject is then detected and alterations in the phenotype after administration of the test agent are indicative of therapeutic agents. The alteration of the phenotype may be detected by comparison to a control. Suitable controls include the same subject before administering the test agent, comparison to a control subject lacking a disruption in nArgBP2 or subject without the condition. A test agent may then be identified as a therapeutic agent for the condition if it ameliorates the condition or causes the phenotype to change, e.g. diminish in severity and become more similar to that in a control subject without the condition.

A variety of subjects are useful in the methods described herein. Generally, subjects are mammals. Suitable subjects include, but are not limited to organisms which are within the orders Carnivora (e.g., dogs and cats), Rodentia (e.g., mice, guinea pigs, and rats), and Primates (e.g., humans, chimpanzees, and monkeys). In some embodiments, the subjects are humans. In other embodiments the subject is a mouse, such as the transgenic KO mouse described herein.

The phenotypes associated with the conditions include, but are not limited to, the level of activity of the subject, compulsive behavior level of the subject, repetitive behavior of the subject, weight gain of the subject, risk-taking behavior likelihood of the subject, hedonistic behavior by the subject, fearlessness of the subject, irritability of the subject, psychotic behavior of the subject, circadian patter of the subject, and anti-depressant activity of the subject.

Compounds or test agents produced or identified as therapeutic agents by application of the assay procedures described herein to the test agents are useful in vitro and in vivo as a treatment for bipolar disorder. Subjects that can be treated by the compounds identified by the methods disclosed herein include, but are not limited to, humans and animals (e.g., dogs, cats, horses, cattle) for veterinary purposes.

As noted above, test agents and therapeutic agents which are active compounds produced or identified by the methods described herein and pharmaceutical formulations of the same (e.g., said compound in a sterile pyrogen-free saline solution), along with the use of such compounds for the preparation of a medicament for the treatment of a nArgBP2 disorder either alone or in combination with other compositions may be used to treat bipolar disorder. The formulations can be used to treat subjects as an active compound or can be used as test agents for the identification of active compounds.

The test and therapeutic agents described above can be combined with a pharmaceutical carrier in accordance with known techniques to provide a pharmaceutical formulation useful carrying out the methods described above. See e.g., Remington, The Science And Practice of Pharmacy (9th Ed. 1995; Mack Publishing Co., Easton, Pa.). In the manufacture of a pharmaceutical formulation according to the presently disclosed subject matter, the active agent (including the physiologically acceptable salts thereof) is typically admixed with, inter alia, an acceptable carrier. The carrier must, of course, be acceptable in the sense of being compatible with any other ingredients in the formulation and must not be deleterious to the patient. The carrier can be a solid or a liquid, or both, and in some embodiments is formulated with the compound as a unit-dose formulation, for example, a tablet, which can contain from 0.01 or 0.5% to 95% or 99% by weight of the active compound. One or more active agents can be incorporated in the formulations, which can be prepared by any of the well known techniques of pharmacy such as admixing the components, and optionally including one or more accessory ingredients.

The formulations of the test agents or therapeutic agents include those suitable for oral, rectal, topical, buccal (e.g., sub-lingual), parenteral (e.g., subcutaneous, intramuscular, intradermal, or intravenous), and topical (i.e., both skin and mucosal surfaces). The most suitable route in any given case will depend on the nature and severity of the condition being treated and on the nature of the particular active compound which is being used.

Formulations suitable for parenteral administration comprise sterile aqueous and non-aqueous injection solutions of the test or therapeutic agents(s), which preparations are in some embodiments isotonic with the blood of the intended recipient. These preparations can contain anti-oxidants, buffers, bacteriostats and solutes which render the formulation isotonic with the blood of the intended recipient. Aqueous and non-aqueous sterile suspensions can include suspending agents and thickening agents. The formulations can be presented in unit-dose or multi-dose containers, for example sealed ampoules and vials, and can be stored in a freeze-dried (lyophilized) condition requiring only the addition of the sterile liquid carrier, for example, saline or water-for-injection immediately prior to use. Extemporaneous injection solutions and suspensions can be prepared from sterile powders, granules, and tablets. For example, in one aspect of an injectable, stable, sterile composition, or a salt thereof is provided in a unit dosage form in a sealed container. The compound or salt is provided in the form of a lyophilizate which is capable of being reconstituted with a suitable pharmaceutically acceptable carrier to form a liquid composition suitable for injection thereof into a subject.

The unit dosage form typically comprises from about 10 mg to about 10 grams of the compound or salt. When the compound or salt is substantially water-insoluble, a sufficient amount of emulsifying agent which is physiologically acceptable can be employed in sufficient quantity to emulsify the compound or salt in an aqueous carrier.

Formulations suitable for topical application to the skin can in some embodiments take the form of an ointment, cream, lotion, paste, gel, spray, aerosol, or oil. Carriers which can be used include petroleum jelly, lanoline, polyethylene glycols, alcohols, transdermal enhancers, and combinations of two or more thereof.

Formulations suitable for transdermal administration can be presented as discrete patches adapted to remain in intimate contact with the epidermis of the recipient for a prolonged period of time. Formulations suitable for transdermal administration can also be delivered by iontophoresis and typically take the form of an optionally buffered aqueous solution of the test or therapeutic agent. Suitable formulations may comprise citrate or bistris buffer (pH 6) or ethanol/water.

Formulations suitable for oral administration may be presented in discrete units, such as capsules, cachets, lozenges, or tablets, each containing a predetermined amount of the test or therapeutic agent; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations are prepared by uniformly and intimately admixing the test or therapeutic agent with a liquid or finely divided solid carrier, or both, and then, if necessary, shaping the resulting mixture. For example, a tablet may be prepared by compressing or molding a powder or granules containing the test or therapeutic agent, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Methods for assessing the risk of an individual developing a disorder associated with aberrant production of the nArgBP2 protein or polypeptide are disclosed. In this method an individual is evaluated to determine the nArgBP2 genotype or the expression level of nArgBP2. The individual may be a human and may be suspected of having bipolar disorder, schizophrenia or autism. The levels of nArgBP2 expression and the nArgBP2 genotype may be evaluated using a tissue sample, group of cells, single cell or the like obtained from the individual. The cells may then be subjected to a quantitative detection of the amount of nArgBP2 protein in the sample from the individual. Alternatively, the genotype of the nArgBP2 alleles may be evaluated using standard techniques as described in more detail below. The nArgBP2 genotype or the expression level of nArgBp2 polypeptide is then compared with that found in controls. Suitable controls include individuals lacking a disruption in nArgBP2, individuals having normal expression of nArgBP2 and individuals displaying normal phenotypes. A significant or substantial difference in the nArgBP2 genotype or the nArgBP2 polypeptide levels as compared to a healthy control that is not expressing a nArgBP2 associated phenotype is indicative of the risk of developing a clinical disorder.

Tissues suitable for biopsy in the above-described methods include tissues in which nArgBP2 is normally expressed. Such tissues include, but are not limited to, brain tissue and peripheral nerve tissue. The nArgBP2 genotype may be evaluated in any cell type obtained from the individual.

The level of nArgBP2 expression in the biopsied tissue or cells can be quantitatively detected by a number of techniques including methods known to those skilled in the art. Techniques which quantitatively detect the protein product include but are not limited to, immunoassays (e.g., ELISA, Western blot analysis, and immunofluorescence). Antibodies which specifically recognize the nArgBP2 protein are known or may be generated by the skilled practitioner. Certain immunoassay-based techniques also provide information regarding the protein product, (e.g., size and subcellular localization) which can also serve as an indicator of aberrant nArgBP2 expression or function.

Techniques that quantitatively detect the amount of nArgBP2 mRNA are also useful in determining the level of expression of nArgBP2. Such techniques are known to those skilled in the art and may be performed using hybridization-based assays (e.g., RNA or Northern blot analysis, reverse transcriptase polymerase chain reaction (RT-PCR). Such assays may be utilized to determine RNA size and sequence, which can serve as an indicator of aberrant expression or protein function or an aberrant genotype. Other assays can potentially identify a mutant nArgBP2 allele which is expressed at normal levels but is impaired in function.

One technique for analysis of the nArgBP2 genotype is PCR amplification of one or more regions of the nArgBP2 gene, followed by size analysis of the amplified product. A detectable difference in size of the amplified product, as compared to that from identically amplified wild type gene, is an indication of the presence of a mutation. A mutation which alters the size of the amplified product is likely to alter nArgBP2 expression or function, and should be further analyzed (e.g., by sequencing the amplified region). Some mutations which affect gene expression or protein function might not alter the size of the amplified region, and must be identified by other techniques, such as direct DNA sequencing or restriction fragment length polymorphism analysis (RFLP). Regions that can be targeted for amplification include, but are not limited to, coding regions of the nArgBP2 gene (e.g., exons). As mutations which affect expression and function have also been known to occur in non-coding regions, examination of non-coding regions (e.g., introns, promoters) can also be performed.

Methods for treating an individual diagnosed with a nArgBP2 disorder by administration of a nArgBP2 enhancer are also disclosed herein. Restoration of a functional nArgBP2 gene in such an individual is expected to have a therapeutic effect. Such restoration can be achieved by administering to the individual a therapeutically effective amount of an enhancer of a nArgBP2 activity to ameliorate the disorder. Enhancers of nArgBP2 include any agent capable of enhancing expression of nArgBP2 and include, but are not limited to small molecules, nucleic acids, proteins and polypeptides. For example, a therapeutically effective amount of nArgBP2 polypeptide can be administered to the individual. Alternatively, the nArgBP2 activity can be increased by introducing an expression construct containing a nucleic acid encoding nArgBP2 operably linked to a promoter into cells of the individual under conditions appropriate for expression.

Cells of the individual appropriate for targeted introduction of an expression vector are cells known to naturally express the wild type nArgBP2, as discussed above. In the absence of restoring wild type nArgBP2 expression to all such cells in the individual, restoration of wild type nArgBP2 expression in a subset of these cells is expected to provide significant therapeutic benefit. Expression vectors currently known in the art and suitable for use in the above described method include, but are not limited to, adenovirus-based expression vectors, lentivirus-based vectors, adeno-associated virus (AAV) based vectors, and herpesvirus based vectors.

### EXAMPLES

### Example 1

### nArgBP2 is Highly Expressed in Brain Regions Implicated in Bipolar Disorder

To determine the expression patterns of nArgBP2 expression in the brain, in situ hybridization was performed to detect mRNA expression of nArgBP2 as previously described (Welch et al., 2004, J. Comp. Neurol. 472:24-39). Briefly, mouse sequences encoding the brain specific exon of the nArgBP2 gene (Genbank accession number NM 172752, nucleotide # 1781-2779 of SEQ ID NO: 2) were used as probe. Digoxigenin (DIG)-labeled RNA probes were synthesized with the MAXIscript in vitro RNA synthesis kit (Ambion, Austin, Tex.) using T3 and T7 RNA polymerases. Fresh-frozen brain sections from adult C57BL/6 mice were used.

For hybridization, sections were fixed with 4% paraformaldehyde (PFA) in phosphate-buffered saline (PBS) solution (150 mM NaCl, 12.1 mM Na₂HPO₄, 2.9 mM KH₂PO₄, pH 7.5) for 10 min at 4° C. and washed 3×3 min with PBS. Sections were then acetylated for 10 min at room temperature in TEA buffer (295 ml H₂O, 4 ml triethanolamine, 0.525 ml 12.1M HCl; mix well and add 0.75 ml acetic anhydride just before use). After washing 3 times with PBS, sections were incubated with hybridization buffer (50% formamide, 5×SSC (750 mM NaCl, 75 mM Na-Citrate), 5×Denhardts solution, 500 μg/ml salmon sperm DNA, 250 μg/ml yeast tRNA) for 2 hrs at room temperature, then with DIG-labeled RNA probes (1-2 mg/ml in hybridization buffer, heat at 70° C. for 10 min to denature, cool on ice) overnight at 70° C. in a chamber humidified with hybridization buffer.

After hybridization, sections were first rinsed 2×5 min with 5×SSC followed by 4×1 hr with 0.2×SSC at 70° C. Sections were then cooled to room temperature and incubated with blocking buffer (10% normal sheep serum, 0.2% blocking reagent, (Roche)) for 1 hr followed by incubation with alkaline phosphatase-conjugated anti-DIG antibody (1:2000, Roche) overnight at 4° C. Sections were washed (4×10 min) with TBS (150 mM NaCl, 10 mM Tris-HCl, pH 7.5) and incubated with color detection buffer (100 mM NaCl, 50 mM MgCl₂, 0.24 mg/ml levamisole, 100 mM Tris-HCl, pH 9.5) for 5 min. Color reactions were developed in the presence of NBT (nitro blue tetrazolium, 0.35 mg/ml) and BCIP (5-bromo-4-chloro-3-indolyl-phosphate, 0.175 mg/ml) in color detection buffer. Color reactions were terminated by incubating sections in TE buffer (1 mM EDTA, 10 mM Tris-HCl, pH 8.0). Sections were mounted with 0.2 μm filtered 90% glycerol/H₂O for imaging.

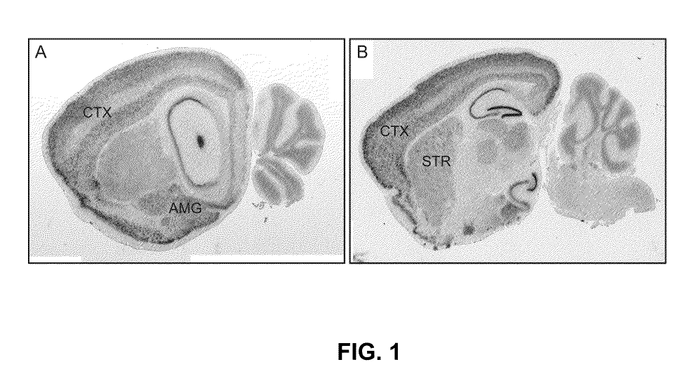

As shown in FIG. 1, nArgBP2 is highly expressed in the cortex, striatum and amygdala (FIG. 1). Dysfunction of these brain regions has been strongly implicated in bipolar disorder as noted above.

### Example 2

### Generation of nArgBP2 Mutant Mice

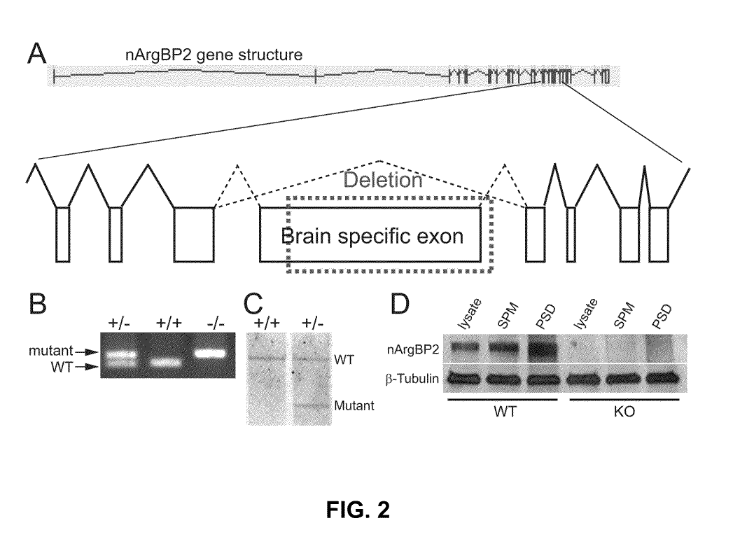

To facilitate the study of the in vivo functions of nArgBP2 at synapses, nArgBP2 gene knockout mice were generated. There are two major alternatively spliced isoforms of nArgBP2. The long form, which contains a large brain-specific exon, is only expressed in the brain, whereas the short form is expressed in non-neural tissues. To specifically dissect the function of nArgBP2 in the brain, the brain-specific long form was disrupted by adding a stop codon in the large brain-specific exon (FIG. 2A). nArgBP2 knockout mice were generated using R1 embryonic stem cells following standard protocols (Welch et al, 2007, Nature 448:894-900). Mutant mice were generated using homologous recombination in mouse ES cells. Deletion of the nArgBP2 gene was confirmed by PCR (FIG. 2B), Southern blot analysis of the genomic DNA (FIG. 2C) and by the lack of detectable corresponding proteins by Western blot (FIG. 2D). Mice homozygous for the nArgBP2 mutation were born at the expected Mendelian rate, grew to the adult stage, and were capable of mating. Anatomical and histological analysis showed that nArgBP2 mutant mice are grossly normal, and no gross structural defects were detected in the brain.

### Example 3

### nArgBP2 Mutant Mice Show Mania-Like Behaviors

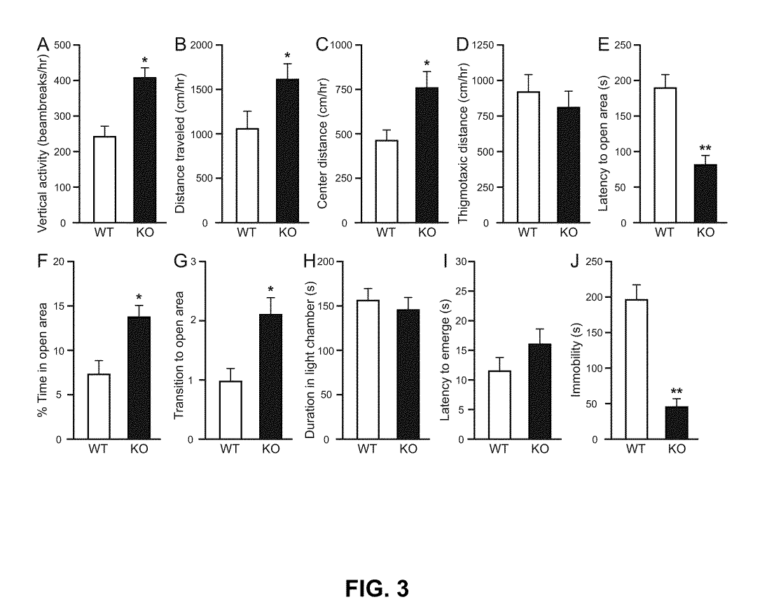

To characterize the phenotypes of nArgBP2 mutant mice, detailed behavioral analysis of these mice were performed. Open filed test, elevated zero maze test, dark-light emergence test, and tail suspension test were performed as previously described (Bakeman and Gottman, 1997, Observing Interaction: An Introduction to Sequential Analyses, Cambridge University Press, New York, pp 56-90; Pogorelov et al., 2005, Neuropsychopharmacology 30, 1818-1831; and Weisstaub et al., 2006, Science 313, 536-40). The nArgBP2 mutant mice show hyperactivity in the open field test as measured by their increased vertical activity as well as increased distance traveled (FIG. 3A-B). These mutant mice also show risk-taking behaviors: in the open field test. They explore center areas more than do wild-type mice, while their activity along the perimeter is the same (FIG. 3C-D). In the elevated zero maze test, nArgBP2 mutant mice show reduced latency to enter the open area, spend more time in the open area, and make more transitions to the open area, again indicating risk-taking behaviors (FIG. 3E-G). However, nArgBP2 mutant mice show similar behaviors in the dark-light emergence test, suggesting that the risk-taking behaviors of nArgBP2 KO mice are not due to the altered level of anxiety (FIG. 3H-I). In addition, nArgBP2 mutant mice show reduced immobility time (struggle longer) in tail suspension test (FIG. 3J), suggesting an anti-depression-like behavior, a key feature of manic behavior.

### Example 4

### SAPBP2 Mutant Mice are Fearless

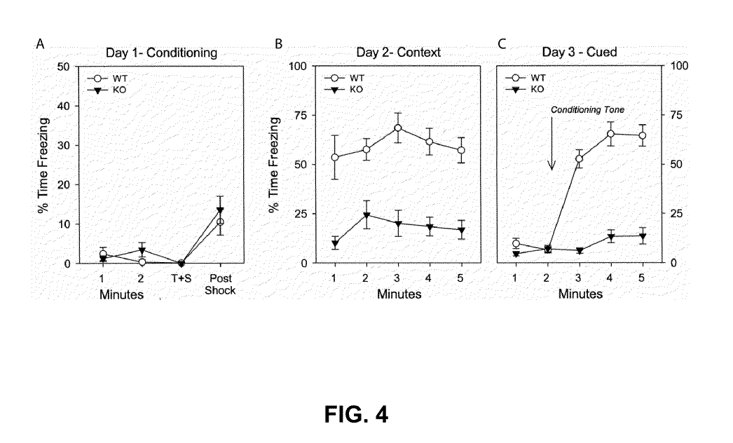

One of the characteristics of mania and hypomania is disinhibited, fearless behavior. The nArgBP2 mutant mice were tested for fearlessness using the fear conditioning test (Welch et al., 2007, Nature 448:894-900) and also show fearless phenotypes. As shown in FIG. 4, nArgBP2 mutant mice are defective in both contextual and cued fear conditioning. This is consistent with potential defects in the amygdale, since nArgBP2 is highly expressed in the amygdala, which plays a key role in emotion and fear.

### Example 5

### Mania-Like Behaviors in nArgBP2 Mutant Mice are Effectively Treated with Lithium

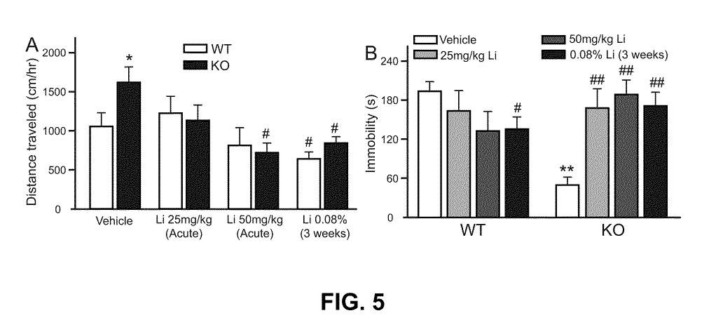

To determine whether the mania-like behavior in nArgBP2 mutant mice can be treated with drugs commonly used for treatment of bipolar disorder, the effects of lithium chloride (LiCl) treatment were tested on the abnormal behaviors of nArgBP2 mutant mice. LiCl is one of the most effective and commonly used drugs for bipolar disorder. Both acute and chronic administrations of LiCl were found effective in treating the mania-like behaviors in nArgBP2 mutant mice. As shown in FIG. 5A, LiCl treatment reduces the activity of the nArgBP2 mutant mice to the level of wild-type mice in the open field test. Similarly, treatment with LiCl normalizes the immobility time of the nArgBP2 mutant mice in the tail suspension test (FIG. 5B). These data further support the conclusion that nArgBP2 mutant mice show mania/bipolar-like behaviors, and that these mutant mice are a valuable tool for developing and testing new treatments for bipolar disorder.

### Example 6

### nArgBP2 Mutant Mice are Obese

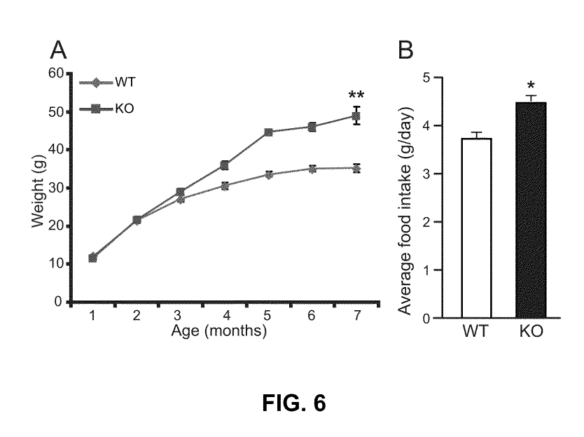

In addition to the above described mania/bipolar-like behaviors, nArgBP2 mutant mice are obese. These mice start to gain excessive weight around 3 months of age even with the regular chow (FIG. 6A). Monitoring their daily food consumption revealed that nArgBP2 mutant mice eat more compared to wild-type littermates (FIG. 6B). This is very interesting because it is well documented that bipolar patients have increased risk of obesity and metabolic syndrome, further supporting that nArgBP2 plays an important role in the pathogenesis of bipolar disorder.

### Example 7

### Altered Synaptic Transmission at Cortico-Striatal Synapses in nArgBP2 Mutant Mice

To survey synaptic defects in nArgBP2 mutant mice, field recordings from cortico-striatal synapses in 21-25 days old mice were obtained. Recordings were obtained in the presence of picrotoxin (GABA_{A} antagonist) to avoid multi-synaptic and contaminating responses from the intra-striatal circuitry. Briefly, 300 μm acute sagittal brain slices from P17-P25 mice were used for all experiments. Recording perfusion solution contained (in mM): 119 NaCl, 2.5 KCl, 1.2 NaH₂PO₄, 26 NaHCO₃, 1 MgCl₂, 2 CaCl₂, and 0.1 picrotoxin (GABA_{A} receptor antagonist). Slicing and recovery solutions were identical to perfusion solution except for containing 0.5 CaCl₂ and no picrotoxin. APV (50 μM) and NBQX (50 μM) were obtained from Tocris (Ellisville, Mo.). Solutions were continuously equilibrated with 95% O₂ and 5% CO₂ (pH 7.4) and perfusion flow rate was 2 ml/min. Slices were allowed to recover for a minimum of 1 hr at 30-32° C. following slicing. All recordings were performed at 30-32° C.

The field recording electrode was placed in the dorso-lateral striatum and a monopolar stimulation electrode was placed in the corpus callosum. Current was delivered to the stimulating electrode using an A.M.P.I. Stimulus Isolator (A.M.P.I., Israel) for 150 μsec. Three distinct components were resolved in the majority of recordings: stimulation artifact, negative peak 1 (NP1, action potential-derived based on latency, resistance to NBQX and picrotoxin, and sensitivity to tetrodotoxin) and negative peak 2 (NP2, fEPSP based on latency and sensitivity to NBQX; in addition, sensitivity to tetrodotoxin indicates response not due to direct activation by stimulating electrode current). The callosal stimulation site was chosen over an intra-striatal site to minimize activation of non-cortical axons. Data were acquired at 20 kHz and filtered at 2 kHz using MultiClamp 700B amplifier and pClamp 10.0 software (Axon Instruments, Sunnyvale, Calif.). Data were analyzed offline using Clampfit 10.0 (Axon Instruments). Five consecutive responses were averaged prior to measuring amplitude, slope or area in the respective assays. When amplitudes are reported, similar conclusions were obtained by slope analysis. Paired-pulse responses were evoked using a stimulation intensity that yielded the maximal fEPSP response. Slope values used for paired-pulse ratios refer to slope during the period from 20-80% of the peak response. NMDAR fEPSPs were evoked using the same stimulation intensity that yielded the maximal fEPSP response under the basal recording conditions used to generate the input-output curves. The area of NMDAR field potential responses was measured during a standard 20 millisecond time window beginning approximately 8 milliseconds after stimulation. All data were collected and analyzed prior to unblinding of genotypes. In addition, NMDAR field potential recordings and correlative PSD biochemical studies were performed in two different labs and were unblinded at the same time.

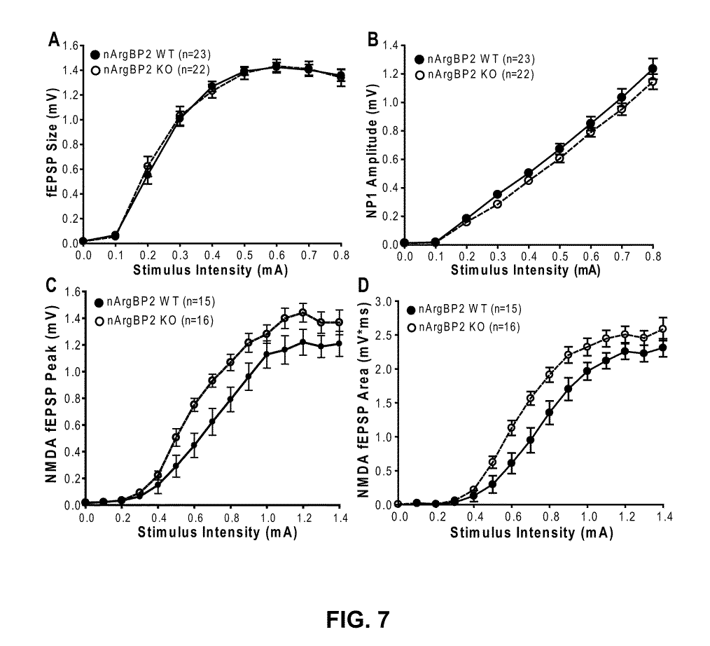

As shown in FIG. 7, total field excitatory postsynaptic potentials (fEPSP) and axonal excitability were not significantly changed in nArgBP2^{-/-} mice in comparison to responses from wild-type littermates (FIGS. 7A, B). NMDAR-mediated responses were monitored by recording in the presence of an AMPAR antagonist (NBQX) and the NMDAR co-factor glycine, and in the absence of magnesium. The NMDAR-dependent fEPSPs were elevated in nArgBP2^{-/-} mice (FIGS. 7C, D), suggesting that nArgBP2 may play an important role in regulating synaptic plasticity. These findings provide a synaptic mechanism as well as a potential therapeutic target for mania-like behaviors.

### Example 8

### Altered Synaptic NMDA and AMPA Receptor Compositions in nArgBP2 Mutant Mice

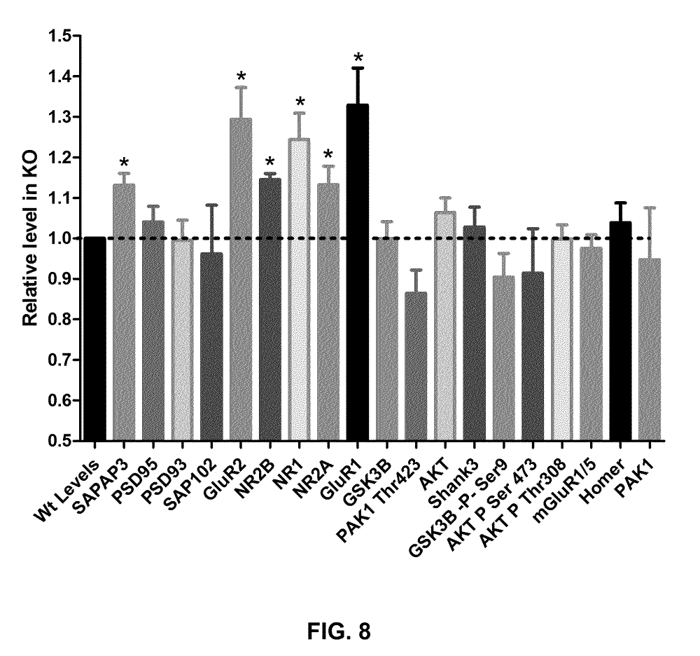

Because nArgBP2 is enriched in the postsynaptic density (PSD) and because electrophysilogical studies indicate an altered synaptic transmission, we investigated whether the composition of synaptic glutamate receptors, which is critical for synaptic transmission, is altered in nArgBP2 mutant mice. Using biochemically purified PSD preparations from adult wild-type (WT) and nArgBP2 mutant (KO) mice, the abundance of various postsynaptic proteins in the PSD was determined by quantitative Western blot analysis (FIG. 8). PSD fractions of the striatum were prepared as described (Welch et al., 2004, J. Comp. Neurol. 472:24-39), separated on SDS-PAGE and probed with specific antibodies. The relative amount of β-tubulin and β-actin were used as loading controls for quantification.

A significant increase of synaptic NMDA receptor subunit NR1, NR2A and NR2B were found in mutant mice, supporting the electrophysiological findings. In addition, a striking increase in synaptic AMPA receptor subunit GluR1 and GluR2 was found. These data indicate that synaptic transmission in adult nArgBP2 mutant mice is significantly increased, which could obviously explain the manic-like behavior and serve as a target for developing more effective treatment. In addition, over-activation of certain synaptic connections could lead to suppression of neuronal activity in other regions of the brain and lead to depression phenotypes.

### Example 9

### Altered Circadian Activity in nArgBP2 Mutant Mice

Ten wild-type and ten mutant mice were each individually housed in a cage equipped with a running wheel and kept within ventilated, light-tight chambers with timer-controlled lighting. Activity was recorded on a computer with the software ClockLab (ActiMetrics Software, Coulbourn Instruments, PA) and analyzed using MatLab (MathWorks, MA) and ClockLab Analysis (ActiMetrics Software, Coulbourn Instruments, PA). Mice were kept in an automated light-dark cycle of 12 hours (LD12:12) for 2 weeks before being switched to a dark-dark cycle (DD) for 3 weeks. When switching to DD, the lights went out at regular circadian time, but did not come on the following day.

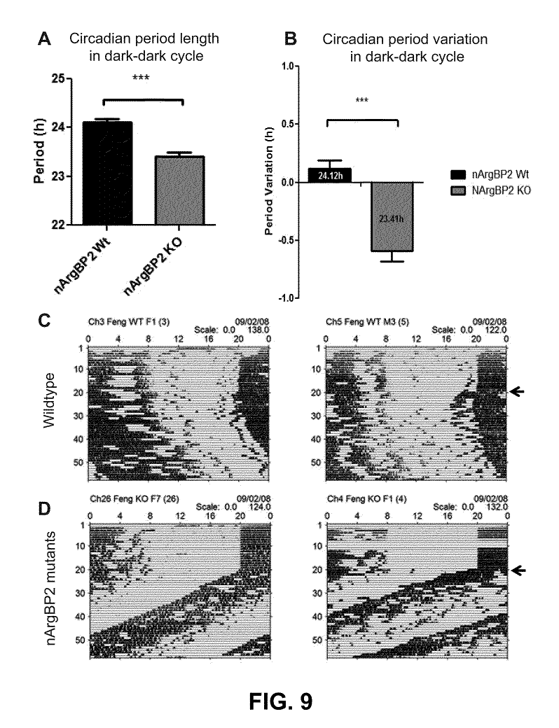

As shown in FIG. 9, when mice were switched to dark-dark cycle (arrows in FIGS. 9C and D) their activity pattern reveals their intrinsic circadian rhythm. FIGS. 9C and D show activity traces of individual mice and demonstrate the dramatically shortened circadian rhythm in nArgBP2 mutant mice as compared to wild-type control mice. FIGS. 9A and B show that nArgBP2 mutant mice have significantly shortened circadian rhythm as compared to wild-type control mice. Because bipolar patients often have circadian rhythm defects, this finding further supports that nArgBP2 plays a role in bipolar disorder.

We claim:

1. A transgenic non-human mammal comprising a disruption in at least one allele of nArgBP2.

2. The transgenic non-human mammal of claim 1, wherein the transgenic non-human mammal is a mouse.

3. The transgenic non-human mammal of claim 1, wherein the disruption comprises a deletion of a portion of nArgBP2 or an insertion into nArgBP2.

4. The transgenic non-human mammal of claim 1, wherein the disruption is in the neuron specific exon of nArgBP2.

5. The transgenic non-human mammal of claim 1, wherein the neuronal cells of the transgenic non-human mammal exhibit reduced expression of a functional nArgBP2 protein.

6. The transgenic non-human mammal of claim 1, wherein the transgenic non-human mammal comprises a disruption of both alleles of nArgBP2.

7. The transgenic non-human mammal of claim 1, wherein the mammal has a phenotype distinct from that of a non-human mammal of the same species lacking a disruption in an allele of endogenous nArgBP2.

8. The transgenic non-human mammal of claim 7, wherein the phenotype includes at least one of increased activity, compulsive behavior, risk-taking behavior, hedonistic behavior, obesity, fearless behavior, psychosis, repetitive behavior, irritable behavior, altered circadian pattern and anti-depressant-like behavior.

9. A neuronal cell comprising a disruption in at least one allele of nArgBP2.

10. The neuronal cell of claim 9, wherein the cell is from a transgenic non-human mammal comprising a disruption in at least one allele of nArgBP2.

11. A method of identifying a therapeutic agent for the treatment of a disorder, the method comprising:
(a) evaluating the level of a nArgBP2 activity or nArgBP2 expression in a cell after contacting the cell with a test agent, wherein the contacted cell has at least one of altered nArgBP2 expression, nArgBP2 activity, SAPAP3 expression, SAPAP3 activity, NMDA receptor expression, NMDA receptor activity, AMPA receptor expression, AMPA receptor activity, p21-activated kinase (PAK) activity, or PAK activity; and
(b) detecting a change in the level of a nArgBP2 activity or nArgBP2 expression in the cell, wherein a change in the level of a nArgBP2 activity or nArgBP2 expression in the cell indicates that the test agent may be a therapeutic agent effective for treating the disorder.

12. The method of claim 11, wherein the cell is present within a transgenic non-human mammal.

13. The method of claim 11, wherein the cell is a neuronal cell or a neuronal cell comprising a disruption in at least one allele of nArgBP2.

14. The method of claim 11, wherein the disorder is selected from the group consisting of bipolar disorder, schizophrenia, and autism.

15. A method of identifying a therapeutic agent for treatment of a condition comprising:
(a) administering a test agent to a subject comprising a disruption of at least one nArgBP2 allele or having altered nArgBP2 expression or nArgBP2 activity, wherein the subject expresses a phenotype associated with the condition; and
(b) detecting a change in the phenotype in the subject, wherein a change in the phenotype is indicative of the ability of the agent to treat the condition.

16. The method of claim 15, wherein the subject is the transgenic non-human mammal comprising a disruption in at least one allele of nArgBP2.

17. The method of claim 15, wherein the phenotype includes at least one of the subject's activity level, level of compulsive behavior, repetitive behavior, weight gain, risk-taking behavior, hedonistic behavior, fearlessness, irritability, circadian pattern and anti-depressant activity.

18. A method of assessing a risk of an individual of developing a disorder associated with a disruption of nArgBP2 comprising:
(a) evaluating the nArgBP2 genotype or the expression of nArgBP2 in the individual; and
(b) detecting an aberrant nArgBP2 genotype or altered level of expression of nArgBP2 in the individual, wherein an aberrant nArgBP2 genotype or altered level of expression of nArgBP2 is indicative of the risk of developing a disorder.

19. The method of claim 18, wherein the individual is suspected of having bipolar disorder.

20. The method of claim 18, wherein the expression of nArgBP2 is evaluated in a sample from the individual and the sample is selected from the group consisting of a cell, a tissue, and a fluid expected to comprise a nArgBP2 polypeptide.

21. The method of claim 18, wherein the expression of nArgBP2 is evaluated in a sample from the individual by obtaining nucleic acids.

22. A method of treating an individual with a disorder associated with reduced nArgBP2 activity comprising administering an effective amount of an enhancer of a nArgBP2 activity to the individual to treat the disorder.

23. The method of claim 22, wherein administering comprises delivering an expression construct encoding a nArgBP2 polypeptide operably linked to a promoter to the individual.

24. The method of claim 22, wherein the enhancer comprises a nArgBP2 polypeptide.
